(19) United States
(12) Patent Application Publication
(10) Pub. No.: US 2008/0095860 A1
(43) Pub. Date: Apr. 24, 2008

(54) EXTRACELLULAR MATRIX TO TREAT MALIGNANCY

(76) Inventor: LEIGH H. FIRESTONE, Berkeley, CA (US)

Correspondence Address: LEIGH HUNT FIRESTONE, 7100 NORFOLK ROAD, BERKELEY, CA, 94705, US

(21) Appl. No.: 11/925,753

(22) Filed: Oct. 27, 2007

Related U.S. Application Data
(63) Continuation-in-part of application No. 11/708,231, filed on Feb. 20, 2007.
(63) Continuation-in-part of application No. 11/865,023, filed on Sep. 30, 2007.
(63) Continuation-in-part of application No. 11/876,963, filed on Oct. 23, 2007.
(60) Provisional application No. 60/775,913, filed on Feb. 22, 2006.

(30) Foreign Application Priority Data
Feb. 21, 2007 (US) PCT/US2007/004332

(51) Int. Cl. A61K 35/37; A61K 35/407; A61P 1/00

(52) U.S. Cl. 424/553; 424/551

(57) ABSTRACT

The invention is articles, compositions and methods for inhibiting growth of an abnormally proliferating cell in a mammal; methods include inhibiting growth of abnormally proliferating cells after tumor resection, and methods of recruiting stem cells to a site of malignancy; articles include sheets of extracellular matrix, and compositions include liquid or semi-solid extracellular matrix compositions having specified concentrations of extracellular matrix.

### CROSS-REFERENCES TO RELATED APPLICATIONS

The present application is a continuation in part to application U.S. Ser. No. 11/708,231 filed Feb. 20^{th}, 2007 which draws priority from U.S. provisional application 60/775,913 filed Feb. 22^{nd}, 2006. The present application also claims priority from PCT application PCT/US2007/004332 filed Feb. 21st, 2007. The present application is also a continuation in part of U.S. Ser. No. 11/865,023 filed Sep. 30, 2007. The present application is also a continuation in part of U.S. Ser. No. 11/876,963 filed Oct. 23, 2007. All related applications are specifically incorporated by reference in their entirety.

### FIELD OF THE INVENTION

The invention is to a compositions and methods of extracellular matrix to treat abnormal cell proliferation in mammalian epithelial tissues and recruit stem cells to a site of malignancy.

### BACKGROUND OF THE INVENTION

Carcinoma is the term for abnormally proliferating and poorly differentiating epithelial cells in mammals. Many types of carcinomas exist, including the very prevalent adenocarcinoma, which describes cancer of the epithelial surfaces of glandular tissues. Breast, colon, lung, thyroid, prostate, stomach, pancreatic, cervical, and ovarian cancers are all examples of tissues at which adenocarcinoma can form. In addition, carcinomas can originate in the bladder, uterus, kidney, lung, skin, and other tissues. This list is not exhaustive.

Types of normal healthy epithelium include simply squamous cells, simple cuboidal cells, simple columnar cells, stratified squamous cells, stratified cuboidal cells, pseudostratified columnar cells and transitional cells. Depending on the organ or tissue, the epithelial cells will take on their appropriate character within these categories.

Tumors are graded or staged depending on their level of differentiation and localization to the site of tumor origin. Staging refers to the condition of the tumor in the context of the surrounding tissues. So for example, Stage I ovarian tumors are confined to one or both ovaries. Stage II ovarian is ovarian cancer that has spread to pelvic organs, but not to abdominal organs. Stage III is ovarian cancer that has spread to abdominal organs. Stage IV is ovarian cancer that has spread outside to distant sites, for example the lung, brain, or lymph nodes in the neck. Within these stages there are subcategories that are identified based on tumor size, node involvement and metastatic status. Thus a tumor can be a IIA, which describes a tumor that has spread and attached to the uterus, where as a IIB tumor describes a tumor that has in addition spread to other pelvic tissues, but with no cancer cells in the ascites or peritoneum, and so on. In addition to tumor staging, epithelial tumors can also be graded. Grade refers to the character of the cells of the tumor. Grade 1 is the least malignant with well-differentiated cells, Grade 2 is intermediate with moderately differentiated cells, and Grade 3 is the most malignant with poorly differentiated cells. Low grade tumors grow more slowly and patients with them have a better prognosis for survival. Analogous staging and grading exists for all carcinomas and adenocarcinomas for every location in the body where the cancers can form.

Further continuing with the ovarian model for exemplary purposes, treatment of epithelial cancers usually involves a surgical first step especially if the tumor is Stage 1A or Stage 1B. Surgery can include hysterectomy (removal of the uterus), bilateral slpingectomy (removal of both fallopian tubes), bilateral oophorectomy (removal of both ovaries), and omenectomy (removal of part of the ornamentum, which is fatty tissue from the upper part of the abdominal cavity near the stomach and intestines). Pelvic and aortic lymph nodes may be sampled and the linings of the pelvis and abdominal cavities can be biopsied to determine if the cancer has spread. If the tumor is deemed a grade 1 or 2, meaning that the cells have some similarities to normal cells, surgery alone may be a cure for the patient. Even if the cancer has not spread, but turns out to be a grade 2 or 3 cell, then chemotherapy may be recommended after surgery. If the cancer is Stage II, then the standard protocol is to “debulk” the tumor in the pelvis as much as possible, which means to remove as much tumor tissue as can be located and safely removed. The protocol for Stage III and Stage IV are the same as for Stage II, with initial surgical treatment followed by chemotherapy. In some Stage III and Stage IV situations, the surgeon will administer chemotherapy directly to the tissues intraperitoneally, and patients have been found to survive longer with this aggressive approach. Follow-up surgery can include laparoscopy to determine if any tumors have regrown in the area of original surgery. Tumor recurrence can also be deduced from blood marker tests, palpation, positron emission tomography, CT scans, and magnetic resonance imaging (MRI).

A survey of the occurrence of carcinoma worldwide indicates that about 400,000 people die a year from carcinoma, and at least about 3 to 4 times that receive an initial diagnosis each year of carcinoma.

It would be a great triumph for medicine and world health if a composition could be developed that lowered the risk of death by cancer in carcinoma patients by providing an effective treatment that significantly prolonged their lives.

### SUMMARY OF THE INVENTION

The invention is a composition comprising a therapeutically effective amount of semi-solid or liquid mammalian extracellular matrix at a concentration less than about 10 mg/ml. The invention can also be a composition comprising a therapeutically effective amount of semi-solid or liquid mammalian extracellular matrix at a concentration greater than about 40 mg/ml. The extracellular matrix of these compositions can comprise a layer of extracellular matrix selected from the group consisting of submucosa, basement membrane, and mucosa.

The invention includes several methods all related to various aspects of treating patients having or at risk for getting carcinoma.

The invention is a method of inhibiting abnormal proliferation of an epithelial cell in epithelial tissue in a patient afflicted with an epithelial cell proliferation disorder, the method comprising: a) contacting said epithelial cell with a composition comprising soft tissue mammalian extracellular matrix; and b) detecting resultant inhibition of cell proliferation in said epithelial tissue in said patient. The extracellular matrix is liquid or semi-solid. The extracellular matrix is a particulate or a sheet. A liquid or semi-solid extracellular matrix is in a concentration greater than about 0.001 mg/ml. The epithelial cell can be malignant.

The invention is also a method of preventing recurrence of an epithelial tumor in a patient comprising: a) removing at least some of an epithelial tumor from a patient forming a resected tumor site, b) contacting said resected tumor site with a composition comprising mammalian soft tissue extracellular matrix, and c) monitoring said resected tumor site for tumor recurrence. The extracellular matrix is liquid or semi-solid. The extracellular matrix is a particulate or a sheet. A liquid or semi-solid extracellular matrix is in a concentration greater than about 0.001 mg/ml. The tumor is stage II or worse.

The invention is also a method comprising: a) providing a composition comprising mammalian extracellular matrix, b) identifying an epithelial tumor in a mammal, said tumor comprising abnormally proliferating epithelial cells, c) disrupting one or more cells in said tumor, forming a disrupted tumor site, and d) contacting said disrupted tumor site with a therapeutically effective amount of said composition. The epithelial tumor is growing in contact with epithelial tissue of tumor origin. The epithelial tumor is stage II or worse. The extracellular matrix is liquid or semi-solid. The extracellular matrix is a particulate or a sheet. A liquid or semi-solid extracellular matrix is in a concentration greater than about 0.001 mg/ml.

The invention is a method of healing a wound in epithelial tissue at a site of excision of an epithelial tumor comprising: a) excising at least a part of an epithelial tumor from a mammal and at least some of a surrounding epithelial tissue forming a tumor excision site, b) contacting said tumor excision site with a therapeutically effective amount of a soft tissue mammalian extracellular matrix, and c) monitoring said site for healing of said surrounding epithelial tissue. The tumor is stage II or worse. The extracellular matrix is liquid or semi-solid. The extracellular matrix is a particulate or a sheet. A liquid or semi-solid extracellular matrix is in a concentration greater than about 0.001 mg/ml.

The invention is a method of inhibiting proliferation of abnormally proliferating epithelial cells in epithelium in a mammal comprising: a) locating a lesion of abnormally proliferating epithelial cells in epithelial tissue, b) excising at least of some of said cells in said lesion and at least some of said epithelial tissue forming a site of tissue disturbance, c) contacting said site of tissue disturbance with a therapeutically effective amount of soft tissue mammalian extracellular matrix, and d) monitoring said site for inhibition of proliferation of said abnormally proliferating epithelial cells. The lesion is stage II or worse. The extracellular matrix is liquid or semi-solid. The extracellular matrix is a particulate or a sheet. A liquid or semi-solid extracellular matrix is in a concentration greater than about 0.001 mg/ml.

The invention is also a method for interrupting abnormal cell growth comprising contacting in vivo an abnormally growing cell with a composition comprising extracellular matrix in a form capable of direct contact with the cell in vivo.

The invention is a method of preventing recurrence of a tumor at a site after removal of the tumor from the site comprising contacting tissue at the site with extracellular matrix in a form capable of in vivo contact with the tissue.

The invention is a method of recruiting endogenous stem cells to a site of abnormally proliferating cells in mammalian tissue comprising: a) locating a site of an abnormally proliferating cell in a tissue type of a mammal; b) contacting said site with a composition comprising mammalian extracellular matrix, and c) observing recruitment of an endogenous stem cell to said site.

The invention is also a method comprising: a) locating a site comprising an abnormally proliferating cell in a tissue in a mammal; b) contacting said site with a therapeutically effective amount of mammalian extracellular matrix; c) recruiting an endogenous stem cell to said site, and d) detecting inhibition of said abnormally proliferating cell in the presence of said recruited stem cells.

The invention is a method of directing differentiation of a poorly differentiated epithelial cell comprising: a) locating a poorly differentiated epithelial cell in epithelial tissue in a mammal; b) contacting said poorly differentiated epithelial cell with a composition comprising mammalian extracellular matrix; c) recruiting an endogenous stem cell to said epithelial tissue in said mammal, and d) observing differentiation of said poorly differentiated cell in said epithelial tissue.

The invention is a composition comprising a therapeutically effective amount of particulate, emulsion exogenous native extracellular matrix in sufficient amount to contact and fill a wound remaining after surgical tumor excision, or an article comprising a sufficient sheet extracellular matrix to contact and fill a wound remaining after surgical tumor excision.

The composition or article includes extracellular matrix that comprises a layer of extracellular matrix selected from the group consisting of submucosa, basement membrane, and mucosa.

The extracellular matrix can be porcine. The extracellular matrix can be bovine. The extracellular matrix can be human.

The extracellular matrix can be small intestine submucosa. The extracellular matrix can be liver basement membrane. The extracellular matrix can be urinary bladder submucosa. The extracellular matrix can be stomach submucosa.

The invention includes a method of inhibiting abnormal proliferation of an epithelial cell in epithelial tissue in a patient afflicted with an epithelial cell proliferation disorder, said method comprising: a) contacting said epithelial cell with a composition or article comprising soft tissue mammalian extracellular matrix; and b) detecting resultant inhibition of cell proliferation in said epithelial tissue in said patient.

The extracellular matrix can be a liquid. The extracellular matrix can be a semi-solid such as an emulsion or gel. The extracellular matrix can be a particulate. The extracellular matrix can be a sheet, or a multilaminate article of several sheets laminated together. The extracellular matrix can be small intestine submucosa. The extracellular matrix can be liver basement membrane. The extracellular matrix can be urinary bladder submucosa. The extracellular matrix can be stomach submucosa. The extracellular matrix can be porcine. The extracellular matrix can be bovine. The extracellular matrix can be human.

The epithelial cell can be malignant. The epithelial cell can be pre-malignant.

The invention includes a method of preventing recurrence of an epithelial tumor in a patient comprising: a) removing at least some of an epithelial tumor from a patient forming a resected tumor site, b) contacting said resected tumor site with a composition or article comprising mammalian soft tissue extracellular matrix, and c) monitoring said resected tumor site for tumor recurrence

The extracellular matrix can be a liquid. The extracellular matrix can be a semi-solid such as an emulsion or gel. The extracellular matrix can be a particulate. The extracellular matrix can be a sheet, or a multilaminate article of several sheets laminated together. The extracellular matrix can be small intestine submucosa. The extracellular matrix can be liver basement membrane. The extracellular matrix can be urinary bladder submucosa. The extracellular matrix can be stomach submucosa. The extracellular matrix can be porcine. The extracellular matrix can be bovine. The extracellular matrix can be human.

### DETAILED DESCRIPTION OF THE INVENTION

The invention is to compositions and methods for treating patients manifesting abnormally proliferating epithelial cells in epithelial tissue. Abnormally proliferating cells form carcinomas which are tumors located at an epithelial surface of the particular organ or tissue of origin. As the carcinoma takes hold at a particular site a single abnormally proliferating cancer cell can become a group of such cells or a tumor or lesion. A tumor of such cells can be recognized graded and staged. Stages typically include Stage IA, IB, IIA, IIB, IIC, III, and IV. Tumor cells are graded from grade 1 (least malignant), grade 2 (intermediate), and grade 3 (the most malignant with poorly differentiated cells). Metastasis begins when cells at the original site break away from the original aggregate of cells and move through the body to attach and proliferate elsewhere. In the beginning the abnormally proliferating cells can be characterized as hyperplastic and somewhat resembling of normal epithelial cells in the region. As the cells progress to carcinoma grade cells and a worse stage of tumor they become malignant, poorly differentiated, and neoplastic, resembling less and less the original epithelial cells of the primary organ. Carcinomas can develop in any epithelial tissue in the body, and when that tissue is a glandular tissue, the cancer is an adenocarcinoma.

The composition of the invention is extracellular matrix in liquid or semi-solid form (e.g. an injectable solution, a gel or an emulsion) at concentrations that have not before been created or used. In one aspect the concentration of liquid or semi-solid extracellular matrix is less than about 10 mg/ml. The concentration can be in a range from about 10 mg/ml to about 0.001 mg/ml. Accordingly, the concentration of this composition can be about any of the following concentrations and those concentration in between these numbers including about 0.001 mg/ml, 0.002 mg/ml, 0.003 mg/ml, 0.004 mg/ml, 0.005 mg/ml, 0.006 mg/ml, 0.007 mg/ml, 0.008 mg/ml, 0.009 mg/ml, 0.01 mg/ml, 0.02 mg/ml, 0.03 mg/ml, 0.04 mg/ml, 0.05 mg/ml, 0.06 mg/ml, 0.07 mg/ml, 0.08 mg/ml, 0.09 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.5 mg/ml, 2.0 mg/ml, 2.5 mg/ml, 3.0 mg/ml, 3.5 mg/ml, 4.0 mg/ml, 4.5 mg/ml, 5.0 mg/ml, 5.5 mg/ml, 6.0 mg/ml, 6.5 mg/ml, 7.0 mg/ml, 7.5 mg/ml, 8.0 mg/ml, 8.5 mg/ml, 9.0 mg/ml, 9.5 mg/ml, and 10.0 mg/ml.

The composition of the invention is also an extracellular matrix in liquid or semi-solid form (e.g. an injectable solution, a gel or an emulsion) at a concentration greater than about 40 mg/ml. Accordingly, the concentration of extracellular matrix in liquid or semi-solid form can be in a range from about 40 mg/ml to about 200 mg/ml, and can include any of the values in between these numbers in the range, including, for example the following numbers and also values in between these numbers such as about 40 mg/ml, 45 mg/ml, 50 mg/ml, 55 mg/ml, 60 mg/ml, 65 mg/ml, 70 mg/ml, 75 mg/ml, 80 mg/ml, 85 mg/ml, 90 mg/ml, 95 mg/ml, 100 mg/ml, 105 mg/ml, 110 mg/ml, 115 mg/ml, 120 mg/ml, 125 mg/ml, 130 mg/ml, 135 mg/ml, 140 mg/ml, 145 mg/ml, 150 mg/ml, 155 mg/ml, 160 mg/ml, 165 mg/ml, 170 mg/ml, 175 mg/ml, 180 mg/ml, 185 mg/ml, 190 mg/ml, 195 mg/ml, and 200 mg/ml.

The extracellular matrix for the compositions and methods is from a mammalian source and can be any extracellular matrix from any tissue in a mammal. Mammals can include humans, horses, monkeys, cows, pigs, sheep, dogs, rabbits, rodents, and generally any otherwise healthy mammal. The extracellular matrices can be from any mammalian tissue having an extracellular matrix, particularly matrices that support soft tissue, and not hard tissue like enamel. Enamel matrices are excluded from the compositions of the invention. The extracellular matrices can be from for example, small intestine, liver, urinary bladder, stomach, pancreas, placenta, large intestine, heart, lung, kidney, and in general any tissue in the mammalian body. Fetal extracellular matrices can be used from any fetal organ of any mammal. It is also possible that other animals, including fish and birds may provide extracellular matrix of sufficient quality for use in the compositions. Additives can be mixed into the extracellular matrix. Additives can include such molecules as immunotherapeutic molecules, cells, anticancer agents, nucleic acids, peptides, polypeptides and proteins.

The extracellular matrix can be solid, semi-solid or liquid. The solid extracellular matrixes can be a sheet, a particulate, a small piece, patch, strip, pellet, plug, strand, weave, or any other form of solid extracellular matrix suited to the task of contacting the abnormal cells or the tissue at the resected tumor site in vivo. Preferred forms are sheets or particulate. Particulate can be made from drying sheets and breaking them up in to fine powder that can be stored, reconstituted, used as is, or delivered in a particular mode, for example by spray or dusting.

Extracellular matrix is a generic term for the proteinous material that exists outside of cells in tissues, supporting cells and cell-protein interactions, among many other functions. Extracellular matrix from epithelial tissues typically has a basement membrane layer, a submucosal layer, and a mucosal layer. Some tissues have an interstitial layer, lamina propria, tunica propria and other layers of matrix. Many of these layers are distinct for particular tissues, and some layers have synonymous terms in the same or different tissues. The invention contemplates any and all of these layers or divisions of matrix, either all together, or separately, or in various combinations to form the compositions of the extracellular matrix. It is generally believed that the submucosal layer is the most active and important of the extracellular matrix layers, and as such, preferred compositions and methods include at least a submucosa in the composition. Submucosa as derived from some mammalian tissues is described in U.S. Pat. No. 4,902,508, U.S. Pat. No. 5,281,422, U.S. Pat. No. 5,281,422, U.S. Pat. No. 5,275,826, U.S. Pat. No. 5,554,389, and other related US patents. Liquid extracts of these matrices and how to make them are described in U.S. Pat. No. 6,375,989 and U.S. Pat. No. 6,579,538, describing how to make liquid and semi-solid extracellular matrix compositions. All of these patents and any related commonly owned patents and patent applications with supportive disclosure are hereby incorporated by reference in their entirety. Generally to make the extracts of matrices, sheets are prepared from the tissues and lyophilized, and then broken up into a fine powder that can be reconstituted in saline or other suitable buffer at a desired concentration.

The invention is an article or composition comprising mammalian extracellular matrix (ECM) for placing in the space remaining after a tumor excision in epithelial tissue in which both a tumor and tissue next to the tumor are excised. The article can be a sheet or several sheets of ECM. The sheets can be placed in the excised tumor space, crumpled into balls, or wads and placed in the space in the breast, or rolled loosely or tightly and configured to fit into the space left after the tissue and tumor have been excised. The composition is particulate ECM or emulsion or gel ECM.

Accordingly, the particulate, emulsion or gel compositions can comprise ECM and other materials as well. Optimally, the composition comprises only ECM, and the ECM been processed so as to retain key growth factors and other molecules and proteins so that when the ECM is placed in the body, the ECM remodels to become the host tissue with which it is in contact. Other materials added to the ECM could be, for example, a therapeutic agent, a drug, added proteins or added cells.

Natural ECM materials suitable for use with the present invention include mammalian small intestine submucosa (SIS), stomach submucosa (SS), urinary bladder submucosa (UBS), dermis, or liver basement membranes (LBM) derived from sheep, bovine, porcine or any suitable mammal. Small intestine submucosa (SIS) is described in U.S. Pat. Nos. 4,902,508, 4,956,178, and 5,275,826; urinary bladder submucosa (UBS) is described in U.S. Pat. No. 5,554,389, stomach submucosa (SS) is described in U.S. Pat. No. 6,099,567, and liver submucosa (LS) or liver basement membrane (LBM) is described in U.S. Pat. No. 6,379,710. See also U.S. Pat. No. 5,554,389, U.S. Pat. No. 4,902,508, and U.S. Pat. No. 5,281,422. All of these patents and any related commonly owned patents and patent applications with supportive disclosure are hereby incorporated by reference in their entirety.

Although these particularly named extracellular matrices are known and have been isolated and used, there may be other mammalian tissues from which extracellular matrix can be isolated and prepared and as such would be suitable for the purposes of the invention. Extracellular matrix-like materials are also generally described in the article “From Cell-ECM Interactions to Tissue Engineering”, Rosso et al, Journal of Cellular Physiology 199, 174-180 (2004). Enamel matrices, which are the extracellular matrix in the tissue around forming teeth, are described in U.S. Pat. No. 7,033,611. The disclosures of all references cited herein are incorporated in their entirety by reference. Extracellular matrices from these tissues have been isolated, processed to retain key growth factors and structural molecules, and dried to become solid materials (sheets and particulates). Particulate forms can be rehydrated in a suitable buffer to become fluidized or emulsion or gel forms. Presently, these extracellular matrix articles and compositions are used for tissue grafting, wound healing, and tissue regenerative purposes.

The invention proposes use of these ECM articles and compositions and materials and forms for placement in the space remaining after a tumor excision as a surgical procedure for the purpose of reducing a likelihood of tumor recurrence in the at the site of tumor excision. The ECM articles and compositions and materials and forms are also proposed in order to regenerate the tissue lost from the organ or tissue during the tumor excision. In addition, the ECM articles and compositions and materials and forms are also proposed in order to reduce localized scarring that can result from the surgical wound created by the surgical procedure.

Methods of use of the articles and compositions of the invention are also contemplated, for example as part of a routine tumor removal or debulking procedure to excise an epithelial tumor or carcinoma, and replace the space in the organ or tissue with mammalian ECM in a sheet form (i.e. as an article) or as a particulate or emulsion or gel form (i.e. as a composition). Surgical excision of the tumor, and placement of the ECM in the space in the organ or tissue after the excision is followed by closing the surgical wound with the ECM article or composition in the breast. Tissue regeneration or wound healing occurs within about 3 to 6 months post surgery.

Mammalian tissue sources are in general any tissue having an extracellular matrix that can be isolated from a mammal and de-cellularized. Thus for example, most mammalian organs are tissue sources. The tissue sources can be for example any mammalian tissue, including but not limited to the small intestine, large intestine, stomach, lung, liver, kidney, pancreas, placenta, heart, bladder, prostate, tissue surrounding growing tooth enamel, tissue surrounding growing bone, and any fetal tissue from any mammalian organ. The decellularization process is important as the material needs to be without its native cells, but the process of the removing the cells need not be so stringent as to remove key active growth factors that contribute to the material's usefulness in the human body. Processes for isolated extracellular matrix from tissues are known in the art, as are processes of decellularizing these matrices.

Extracellular matrix can be obtained from the tissues of mammals by processes such as described in U.S. Pat. No. 5,554,389, U.S. Pat. No. 4,902,508, and U.S. Pat. No. 5,281,422. For example, the urinary bladder submucosa is an extracellular matrix that has the tunica mucosa (which includes the transitional epithelial layer and the tunica propria), a submucosal layer, 3 layers of muscularis, and the adventitia (a loose connective tissue layer). This general configuration is true also for small intestine submucosa (SIS) and stomach submucosa (SS). Obtaining enamel matrices is described in U.S. Pat. No. 7,033,611. Enamel matrix is extracellular matrix existing near forming teeth.

Natural ECM materials include mammalian small intestine submucosa (SIS), stomach submucosa (SS), urinary bladder submucosa (UBS), dermis, or liver basement membranes (LBM) derived from sheep, bovine, porcine or any suitable mammal. Small intestine submucosa (SIS) is described in U.S. Pat. Nos. 4,902,508, 4,956,178 and 5,275,826; urinary bladder submucosa (UBS) is described in U.S. Pat. No. 5,554,389, stomach submucosa (SS) is described in U.S. Pat. No. 6,099,567, and liver submucosa (LS) or liver basement membrane (LBM) is described in U.S. Pat. No. 6,379,710. In the preparation process, native extracellular matrices are prepared so that their bioactivity is preserved, including many cellular and transcriptional and translational event. Assays for determining these activities are standard in the art.

Many of these ECM compositions are generally comprised of the same tissue layers and are prepared by the same method, the difference being that of the starting material (i.e. from one organ versus another). The matrices are generally decellularized in order to render them non-immunogenic, a process that needs to also retain some function of key proteins, such as some growth factors. Specific procedural steps are further detailed in the patents referenced above.

Examples of a typical epithelium having a basement membrane include tissues that have an epithelium such as the skin, intestine, urinary bladder, esophagus, stomach, cornea, and liver. The epithelial basement membrane may be in the form of a thin sheet of extracellular material contiguous with the basilar aspect of epithelial cells. Sheets of aggregated epithelial cells of similar type form an epithelium. Epithelial cells and their associated epithelial basement membrane may be positioned on the luminal portion of the tunica mucosa and constitute the internal surface of tubular and hollow organs and tissues of the body. Connective tissues and the submucosa, for example, are positioned on the abluminal or deep side of the basement membrane, and can include for example the submucosa of the intestine (SIS) and urinary bladder (UBS), and the dermis and subcutaneous tissues of the skin. Typically the material is rinsed with saline and optionally stored in a frozen hydrated state until used.

In addition to employing sheet ECMs to form the articles of the present invention, the ECM material may be fluidized or emulsified. Fluidized UBS, for example, can be prepared in a manner similar to the preparation of fluidized intestinal submucosa, as described in U.S. Pat. No. 5,275,826. The UBS is comminuted by tearing, cutting, grinding, shearing or the like. Grinding the UBS in a frozen or freeze-dried state is preferred although good results can be obtained as well by subjecting a suspension of submucosa pieces to treatment in a high speed (high shear) blender and dewatering, if necessary, by centrifuging and decanting excess water. Additionally, the comminuted fluidized tissue can be solubilized by enzymatic digestion of the bladder submucosa with a protease, such as trypsin or pepsin, or other appropriate enzymes for a period of time sufficient to solubilize said tissue and form a substantially homogeneous solution.

Other examples of ECM material suitable for use with the present invention include but are not limited to dermal extracellular matrix material, subcutaneous extracellular matrix material, large intestine extracellular matrix material, placental extracellular matrix material, ornamentum extracellular matrix material, heart extracellular matrix material, and lung extracellular matrix material, may be used, derived and preserved similarly as described herein for the SIS, SS, LBM, and UBM materials. Other organ tissue sources of basement membrane for use in accordance with this invention include spleen, lymph nodes, salivary glands, prostate, pancreas and other secreting glands. In general, any tissue of a mammal that has an extracellular matrix can be used for developing an extracellular matrix component of the invention.

Other tissues such as the liver and pancreas have a basement membrane that does not demonstrate the kind of tensile strength of the tissues defined as submucosa. However, other useful properties may be opportunistically employed from the extracellular matrices of such tissues as the liver, pancreas, placenta and lung tissues which have either basement membrane for extracellular matrix or interstitial membrane (as with the lung). These softer matrices support cells such as those in the organs from which the matrices are derived. Thus, certain benefits are to be found in using the extracellular matrices of these tissues, especially in combination with other such matrices like SIS and SS that may be stronger and which offer their particular advantages. Accordingly, any of these mammalian matrices can be used with potential effectiveness in certain tissues or organs having carcinoma. Accordingly, the liver, lung, and pancreatic extracellular matrices may be quite suitable for generating some of the sheets, strips or pieces of the articles of the invention, or particulates or gels and may be used as such, such as, for example articles or compositions to be placed in organs or tissues after tumor removal such as liver tumors, lung tumors, and pancreatic tumors.

The article of extracellular matrix can comprise extracellular matrix combinations from such sources as, for example but not limited to, small intestine submucosa, liver basement membrane, stomach submucosa, urinary bladder submucosa, placental basement membrane, pancreatic basement membrane, large intestine submucosa, lung interstitial membrane, respiratory tract submucosa, heart extracellular matrix, dermal matrix, and in general extracellular matrix from any mammalian fetal tissue. Any one of these tissue sources can provide extracellular matrix that can then be manipulated into a designated form (e.g. sheet, strip or piece) for use in the articles of the invention, or particulate or emulsion or gel for the compositions of the invention.

The articles of the invention that are made of sheets, strips, or pieces of extracellular matrix can be made from a single source of extracellular matrix. The composition can also be made from two or more extracellular matrices isolated from a donor mammal or from a particular tissue source in that donor or multiple donors. In any event, the key factor is that at least two tissue sources from which the composition comprising mammalian extracellular matrix can be derived to form the composition derived from different tissue sources.

One method of the invention is inhibiting abnormal proliferation of an epithelial cell in epithelial tissue in a patient afflicted with an epithelial cell proliferation disorder. The epithelial cell proliferation disorder will typically be carcinoma, pre-carcinoma, neoplasia, pre-cancer, cancer, epithelial cell cancer, or any known synonyms or near synonyms for the same. The cells can be hyperplastic, displastic, pre-malignant, malignant, moderately differentiated, poorly differentiated, or the like. Abnormal proliferation is manifest in cells that proliferate. Normal healthy cells do not proliferate once they have terminally differentiated into a tissue type. Cancer cells characteristically manifest abnormal proliferation and therefore grow in clumps that become visible tumors. The cell or cells will be located at a tissue of origin, thus an epithelial tissue, and the cell or cells will generally be proliferating abnormally in the context of an endogenous epithelial extracellular matrix that surrounds and supports the epithelial cells of the region.

In practicing the method, the abnormally proliferating epithelial cell or cells are contacted with a composition of a soft tissue mammalian extracellular matrix. After a passage of some time to allow the composition to have an effect, inhibition of cell proliferation of this cell or cells is detected. It is anticipated that fewer abnormally proliferating cells will be detected than previously existed at the site before contact with the composition, or that the abnormal appearance of a particular cell will have altered to have it appear normal or nearly normal again.

Inhibition of cell proliferation in the epithelial tissue in the patient can be manifest by an absence of such abnormally proliferating cells after a period of time, a reduction in quantity of such abnormally proliferating cells, or an improvement in grade (such as greater cell differentiation) in the cells. The most preferred result is the complete absence of such abnormally proliferating cells, and thus that the abnormally proliferating cells are undetectable or no longer detectable at the site.

Another method of the invention is preventing recurrence of an epithelial tumor in a patient. The method is practiced by removing at least some of the epithelial tumor from a patient forming a resected tumor site. After that, the resected tumor site is contacted with a composition comprising mammalian soft tissue extracellular matrix. The site is closed and enough time is allowed for the site to heal. At some time point later, the area is monitored (preferably non-invasively) for signs of regrowth of the tumor. A preferred result is absence of any regrowth of the tumor. The extracellular matrix used to contact the tumor site can be any form of extracellular matrix, but is preferably a form that provides maximal contact with the site, such as a particulate, semi-solid, or liquid extracellular matrix. Particularly in the case of the creation of a tumor cavity with the excision of a tumor, extracellular matrix is used to fill the cavity. Due to its wound healing capabilities, the extracellular matrix can heal the healthy resected tissue, which inevitably gets excised while trying to excise a tumor. The extracellular matrix also serves to redirect growth and differentiation of any of the remaining malignant tissue, to reprogram its course to become normal tissue again.

A method of the invention provides a composition comprising mammalian extracellular matrix from any source, identifying an epithelial tumor in a mammal, the tumor comprising abnormally proliferating epithelial cells, disrupting one or more cells of the tumor or the tissue surrounding the tumor, to form a disrupted tumor site, and contacting the disrupted tumor site with a therapeutically effective amount of the extracellular matrix composition. Disruption of the tumor cells or the tissues surrounding the tumor is accomplished so that a wound area is created at the site. Disruption can be accomplished by scraping, poking, cutting, or otherwise touching the cells of the tumor and surrounding tissue. Upon contact with the extracellular matrix this damaged tissue of epithelial origin will begin to heal, thus altering what would have been an inevitable growth of cancerous tissue overtaking the healthy tissue. Contact of the region with healthy extracellular matrix from any source will redirect the area to heal from both the recent disruption, and the carcinoma.

The tumor targeted by this method is preferably stage II or worse, including stage IIA, IIB, IIC, stage IIIA, IIIB, IIIC, and stage IV. Often tumors at stage II or worse can not be completely resected with clean margins. Therefore disrupting the tumor, and perhaps removing most of it, will allow the composition of extracellular matrix to come into the region as a therapeutic and heal the disrupted, damaged tissue. The disruption of the tumor can involve not removing any tumor, but rather scrapping or cutting the tumor in situ, providing an opportunity for the extracellular matrix composition to contact the tumor cells more completely. Generally, surgeons do not favor disrupting tumor cells without removing them for fear that a disrupted tumor cell will metastasize to another region of the body, but in the case of this method, all tumor cells are covered or coated with extracellular matrix before closing the site by using liquid or semi-solid extracellular matrix to encase the dislodged tumor and prevent the migration of its cells to another location in the body.

The extracellular matrix can be from a soft tissue mammalian extracellular matrix, and is preferably from an epithelial tissue extracellular matrix. The epithelial tumor is growing in contact with epithelial tissue of tumor origin. By disrupting both the tumor and the surrounding epithelial tissue, upon placement at the site of extracellular matrix composition (e.g. a liquid, semi-solid, or solid extracellular matrix) the new exogenous extracellular matrix can begin to heal the epithelial tissue and generate healthy tissue at the site.

A method of the invention is also a method of healing a wound in epithelial tissue at a site of excision of an epithelial tumor. This method is practiced by excising at least a part of an epithelial tumor from a mammal, and at least some of the surrounding epithelial tissue forming a tumor excision site, contacting the tumor excision site with a therapeutically effective amount of a soft tissue mammalian extracellular matrix and monitoring the site for healing of the surrounding epithelial tissue. The monitoring can be done non-invasively, e.g. by a visualization means or using blood markers. The extracellular matrix of the composition can be liquid, semi-solid, or solid matrix. The solid matrix can be a particulate, or a sheet.

Another method of the invention is a method of inhibiting proliferation of abnormally proliferating epithelial cells in epithelium in a mammal comprising locating a lesion of abnormally proliferating epithelial cells in epithelial tissue, excising at least some of these cells in the lesion and at least some of the epithelial tissue forming a site of tissue disturbance. The next step is contacting the site of tissue disturbance with a therapeutically effective amount of a soft tissue extracellular matrix and monitoring said site for inhibition of proliferation of the abnormally proliferating epithelial cells. In this method, the composition comprising extracellular matrix contacts the region of tissue disturbance and inhibits the abnormally proliferating epithelial cells. The inhibition can be monitored non-invasively by visualizing the site periodically after the initial procedure, or measuring blood markers that indicate the particular carcinoma being treated.

It has not been previously appreciated that recruitment of endogenous stem cells to a site of abnormally proliferating cells in mammalian tissue results in inhibition of the abnormal proliferation of the cells. It is known that endogenous stem cells are recruited to a site of tissue remodeling that is directed with the placement of exogenous extracellular matrix at a site. It has not been before appreciated that placement of exogenous mammalian extracellular matrix at a site of malignancy will result in stem cell participation in the inhibition of the malignancy. Thus the invention includes a method of recruiting endogenous stem cells to a site of abnormally proliferating cells in mammalian tissue by contacting an abnormally proliferating cell in a tissue type of a mammal, with a composition comprising exogenous mammalian extracellular matrix (extracellular matrix from another mammal) thereby recruiting one or more endogenous stem cells to the site. Upon recruitment to the site, the stem cells begin to facilitate remodeling of the damaged and diseased tissue, which has a great effect on the course of the malignancy. Malignant cells become less malignant, and eventually become normal cells and normal tissue as a result of the intervention. The stem cells that are recruited are probably adult stem cells, and most likely are multipotent cells, although they may possibly be pluripotent when they are first recruited to the site.

The invention also refines this study with a method comprising: locating a site comprising an abnormally proliferating cell in a tissue in a mammal and contacting the site with a therapeutically effective amount of mammalian extracellular matrix. The contact at the site with the therapeutically effective amount of mammalian extracellular matrix (solid, semi-solid, or liquid extracellular matrix) results in recruiting one or more endogenous stem cells to the site. Sometime after the stem cells have been recruited and the tissue surrounding the damage has begun to remodel, detection of inhibition of the abnormally proliferating cell or cells can be observed, in the presence of the recruited stem cells. The recruited stem cells may be direct actors in the inhibition process, or they may be indirect actors by providing the proper healthy environment including molecular signaling and tissue remodeling for the inhibition to occur eliminating the abnormally proliferating cells. Either mechanism yields a positive result for the patient. Detection of the inhibition of the abnormally proliferating cells can be accomplished by standard visualization techniques (e.g. MRI, CT, PET scans) or by sampling blood marker levels that can indicate a presence or absence of a malignancy, or a reduced amount of malignancy.

Detection of developing carcinoma before tumor excision, recurring carcinoma after excision, or forming scar tissue (or lack thereof) after tumor excision followed by placement of ECM at the site of tumor excision can be accomplished by a novel technology called Magnetic Resonance Elastography (MRE). MRE can be used to determine the stiffness of inelasticity of a particular tissue. Where, for example, fat tissue has a kPa of 4, and healthy tissue has a kPa of 6, cancer (carcinoma) will often have a kPa in the realm of a value of 20. Scar tissue may have a kPa between that of healthy tissue and cancer, or perhaps a value stiffer than that of cancer. The absolute value of various scar tissues in the various tissues and organs of interest for the purposes of this invention can be determined by routine application of MRE to these tissues or organs in patients both having and not having carcinoma or scarring. Basic information on the technology called MRE can be found at Doyley et al. “Thresholds for detecting and characterizing focal lesions using steady-state MR elastography,” Medical Physics, 30(4):495-504, 2003.

A method of directing differentiation of a poorly differentiated epithelial cell is accomplished by locating a poorly differentiated epithelial cell in epithelial tissue in a mammal, contacting the poorly differentiated epithelial cell with a composition comprising mammalian extracellular matrix, recruiting an endogenous stem cell to the epithelial tissue in the mammal, and observing differentiation of the poorly differentiated cell in the epithelial tissue. As discussed earlier, one of the hallmarks of malignancy is the loss of differentiation of the malignant cell that becomes more and more serious as the cell moves from pre-malignant to fully malignant, losing almost all resemblance it once had to a normal healthy cell. In the presence of recruited stem cells at a site, poorly differentiated cells will become gradually better differentiated as they respond to the local signals generated from the recruited stem cells. New tissue remodels and the once poorly differentiated cells become closer and closer to normal clearly differentiated cells in appearance.

The concentration of the liquid or semi-solid extracellular matrix used in any of these methods is greater than about 0.001 mg/ml. Optimally, the concentration is that concentration that minimally will regenerate missing tissue, heal damaged tissue, inhibit abnormal cell proliferation, and prevent tumor recurrence. This concentration is expected to be at least 10 mg/ml, and as much as 40 mg/ml, or greater. The larger the concentration of matrix, the more effective the healing of the tissue at the site, and so at concentrations greater than 40 mg/ml the composition are expected to be optimally therapeutically effective at healing the site, and inhibiting proliferation of abnormally proliferating cells, and inhibiting the reforming of tumor cells at a site of tumor resection. Furthermore, the exogenous extracellular matrix at the site heals the damaged tissue with reduced or absent scar formation. Solid forms of extracellular matrix can be used, including particulate and sheets as well as any other solid form. The particulate can be dusted in a region, the sheet can be affixed to tissue to heal, for example skin tissue in the case of skin cancer, or the outer layer of an internal organ.

The contact made between the extracellular matrix and the abnormally proliferating cell is made in vivo, in the person or animal afflicted with the abnormal cell proliferation disorder. The contact can be made with the abnormally proliferating cell in the tissue that the cell is growing. For example, a surgeon can identify a tumor in a patient and contact the tumor with sufficient extracellular matrix to cover and surround the tumor or aggregate of cancer cells. Alternatively, a group of such abnormally proliferating cells can be removed and the extracellular matrix composition can be applied at the site of tumor removal, to prevent tumor recurrence. Thus the extracellular matrix composition can be applied to tissue after surgical resection of a tumor, before closing the site. For this reason, subcutaneous tumor models are not ideal in which to demonstrate the full scope and potential of the invention. The contact of exogenous extracellular matrix needs to be made not only with the abnormally proliferating cells, but also with the tissue (and extracellular matrix) that the abnormally proliferating cells are proliferating in. This is because the work that the exogenous extracellular matrix can facilitate is directly tied to its ability to set straight and make right the environment that the malignant cells are propagating in. Subcutaneous tumors have cancer cells growing in clumps virtually unconnected to a tissue, and never connected to the tissue type that that the cells came from. Thus that artificial scenario does not allow the exogenous extracellular matrix to accomplish its full work, that of both remodeling tissue at a site of damage and affecting and influencing the course of the malignancy in addition, or as a result.

To accomplish good contact with the tissue at the site, emulsified, injectable, foam, gel, liquid, glue, paste, small piece, patch, strip, pellet, plug, strand, weave, spray, paint, cream or any malleable, tissue-attachable form of the extracellular matrix material can be used. Conceivably, small pieces, small patches, plugs, strips, pellets, or strands of extracellular matrix material can be attached at the site, or placed there and will work to regenerate healthy tissue at the site of tumor resection. In general, any malleable or appropriate tissue-attachable form of extracellular matrix material that can also contact the abnormally growing cells attached to the tissue can be used at the site, the form primarily being determined by the nature of the tissue, the nature of the cancer cells being targeted for growth inhibition, the nature of the resection, and the anticipated needs for healing and tissue regeneration at the site. For example, sheets of extracellular matrix can be used to close wounds at the dermis with a melanoma or other skin cancers. Optimal compositions of extracellular matrix are liquid, semi-solid or solid formulations.

The extracellular matrix can be in a fluid or liquid form, for example an emulsion or otherwise injectable solution. The extracellular matrix can also be in a semi-solid form, for example a gel, foam, glue, paste, or other semi-solid form. The semi-solid forms may be injectable depending on their viscosity, but they should be applicable to the abnormally proliferating cells or the resected tumor space.

The extracellular matrix can also be a solid, for example any solid form including a powder or particulate that can be sprayed or dusted in a region. The extracellular matrix contacts the tissue and is applied to the site as emulsified, injectable, foam, gel, liquid, glue, paste, small piece, patch, strip, pellet, plug, strand, weave, spray, paint, cream and any malleable form. The extracellular matrix can be a sheet to repair an epithelial layer. Depending on the nature of the tissue and site small patches, plugs, strips, pellets, strands or some other such similar fragments or pieces of solid or semi-solid extracellular matrix material can be used effectively at certain sites with certain tissues, depending largely on the architecture of the tissue, and considering how best to introduce the extracellular matrix. The preferred form of the extracellular matrix will be that form, either liquid, semi-solid or solid that provides maximal contact of the matrix with the tissue or cells that are targeted. So, for example, a solution or emulsion or gel of extracellular matrix will fill a closed space and provide ample contact of the matrix with the tissue of the surrounding region.

Where a tumor has been identified in the patient, the tumor can be addressed by contacting the tumor with an aliquot of extracellular matrix sufficient to cover the tumor and contact all the abnormally growing cells possible. The tumor may also be resected and the space left by the removed tumor can be filled with extracellular matrix. The tumor growth can include, for example, any tumor stage. The composition can also be placed at a site of abnormal cell growth, which can cause an interruption of the abnormal cell growth, and a remodeling of the unhealthy tissue to new healthy tissue. Placing extracellular matrix at a site having cancer or precancer cells can serve to eliminate the cancer cells from the local tissue environment and so eliminate their potential for tumor formation in the body. Placing extracellular matrix in the tumor space can ensure that the tumor will not recur at the site of removal, and will additionally help to heal the tissue so that scarring and disfigurement is limited.

Means of placing the extracellular matrix at the site in the body where a tumor has been resected, or cancer cells are believed to exist, can be accomplished by surgically opening the site and accessing the tissue directly, or by percutanous or other minimally invasive access to the site of cancer or the site where cancer is believed to be. Direct injection of the extracellular matrix at the site either of abnormal cell proliferation or tumor resection may be preferred. A catheter may be able to resect the tumor or lesion of abnormal cells and afterwards apply a coating of extracellular matrix in an injectable, emulsion or spray, for example. Solid pieces of extracellular matrix can be sutured, stapled, glued, or otherwise attached at a site. Plugs, pellets or other pieces of extracellular matrix can be placed or attached at the site using an appropriate attachment means, such a glue or suture.

The compositions of the invention are pharmaceutical compositions which mean that they are acceptable for administration in humans and meet the standards required by the FDA. Any exipient used to make the composition, as with the liquid or semi-solid pharmaceutical compositions are made using pharmaceutically acceptable exipients so that the final composition is suitable, safe and effective for use in humans. The pharmaceutical compositions include an extracellular matrix component, and may also include derivatives or active agents related to it, and also possibly additional pharmaceutical agents that complement, or otherwise contribute to the function of the composition as a whole, such as a carrier, exipient, anti-cancer drug or the like.

By an “effective” amount or a “therapeutically effective amount” of a pharmacologically active agent such as the extracellular matrix it is meant that a nontoxic but sufficient amount of the agent is used in the composition to provide the desired effect of facilitating growth inhibition of abnormally proliferating cells, healing of wounded tissue, or inhibition of tumor recurrence.

### EXPERIMENTAL

For these experiments 1-4, mice are selected for testing the invention. The genes of mice are similar to humans and so mice provide a suitable initial animal model for testing cancer treatments. Jackson laboratory has several strains of mice available that are appropriate for experiments involving the invention. Notably, several JAX® mice have been bred for increased tumor incidence: (JAXmice.jaxorg/models/cancer)

A/J 000646

BALB/cByJ (000651)

CBA/CaJ (000654)

In addition several strains have been developed with special specific propensities:

C57BL/6J-Apc^{MIN} (002020)—propensity to develop adenomas

FVB/N—TgN (MMTV neu) 202Mul (002376)—propensity towards mammary tumors

C57 BL/6—TgN (TRAMP) 8247Ng (003135)—propensity to develop prostate tumors

### Example 1

### 000654

As an initial experiment CBA/CaJ (000654) mice are selected. The 000654 mouse has an increased incidence of late onset mammary gland tumors, and also a propensity towards hematomas and lymphomas.

The CBA inbred strain is susceptible to tumor induction after a single subcutaneous injection of methyl cholanthrene. Accordingly, three female 000654 mice are ordered from Jackson Laboratories in Bar Harbor, Me. (JAX labs). All three mice receive an injection of methyl cholanthrene at 8 weeks in a single injection. The mammary glands of each mouse are identified and marked. All mammary glands are injected with sufficient ml of methyl cholanthrene to cause tumors to grow. The mice are observed for tumor development which is diagnosed by palpation. As the tumors develop, the three mice are treated as follows:

Mouse A—tumor removal, closure of the site and continued observation. The tumor tissue is retained for analysis. An aliquot of blood and lymph is extracted to test for evidence of metastasis.

Mouse B—tumor removal, placement of extracellular matrix emulsion at the site of excision and closure of the wound. The tumor tissue is retained for analysis. An aliquot of blood and lymph is extracted to test for evidence of metastasis.

Mouse C—partial tumor excision, leaving some cancer cells at the site, application of extracellular matrix emulsion at the site of excision and closure of the wound. The tumor tissue is retained for analysis. An aliquot of blood and lymph is extracted to test for evidence of metastasis.

Once a tumor is observed in a mouse, the lesion is surgically removed and the tumor tissue is analyzed to confirm the nature and stage of the cancer cells. An emulsion of extracellular matrix material is placed at the site of tumor removal and the incision is closed. The mouse is monitored for a recurrence of the lesion. If other tumors develop in other glands, those are also removed and the site is filled with an emulsified extracellular matrix material.

At 4 to 6 months the mice are sacrificed. The surgical sites of each site of excision are removed and analyzed for tumor cells, tissue quality, and presence of extracellular matrix. The sites are spread on a slide and photographed. The mouse blood and lymph are tested for evidence of metastasis.

### Example 2

### A/J Mice

A/J mice have a propensity to develop lung tumors in response to carcinogens. A/J mice also have a high incidence of mammary adeno-carcinomas in multi-parous females. Four females are purchased and observed for development of either lung or mammary tumors. When tumors develop in the mice, the tumors are removed and the site injected with ECM and closed.

Mouse A tumor removed completely, ECM added, and site closed.

Mouse B tumor removed partially, ECM added and site closed.

Mouse C tumor not removed, ECM added and site closed.

Mouse D tumor not removed, no surgery. Tumor monitored for progression of growth.

### Example 3

### 002020

C57BL/6J-Apc min is a strain of mouse that is highly susceptible to spontaneous intestinal adenoma formation. One hundred percent of C57BL/6J-Apc min heterozygous mice raised on a high fat diet develop in excess of 30 adenomas through the intestinal tract and most die by 120 days of age. A small number of female mice develop mammary tumors.

Mouse A—control—tumors allowed to develop unchecked.

Mouse B, C, D, E, —tumor development observed

After tumor development is established, two groups are created:

3 mice 1. surgical group+ECM after surgery

3 mice 2. surgical group+no ECM after surgery

As many tumors as develop in these mice and are detected are either removed and contacted with ECM, or simply removed. The mice are observed for overall survivability, cancer or cancer-free condition. Blood and lymph are tested for evidence of metastasis.

### Example 4

### 02376

Mice homozygous for the MMTV/neu (rat) transgene are viable and fertile. Focal mammary tumors first appear at 4 months, with median incidence of 205 days. Both virgin and breeder mice develop tumors. Tumors arise as hyperplastic and displastic tumors. Seventy-two percent of tumor bearing mice that lived to 8 months or longer developed metastatic disease to lung.

Female mice are purchased; mice observed for mammary tumor development.

Mouse A control, no surgery, no ECM

Mouse B surgery plus ECM

Mouse C surgery—no ECM

Mouse D inject ECM at tumor site only (without opening the site).

Those mice that survive past 8 months, are observed for lung carcinoma:

Mouse B surgery+ECM at lungs, if lung tumor develops

Mouse C surgery+no ECM

Mouse D inject ECM at the tumor site only.

### Example 5

### Actual Study with Nude Mice Having SUBQ Tumors

Three groups of nude mice from Charles Rivers laboratory were formed from 24 mice. MDA-MB-231 human breast cancer cells were received from ATCC and cultured to confluence for inoculation in the animals subcutaneously. Each mouse was inoculated beneath the skin in the right and left flanks. Two mice in each group using 1 million cells, two mice using 2.5 million cells, two mice using 5 million cells, and 2 mice using 10 million cells. One group was just cancer cells alone. The second group was Matrigel™ plus cells, and the third group was extracellular matrix emulsion from small intestine submucosa with the cancer cells. As of the date of this filing of this patent application. Tumors were detected in the matrigel mice, at 5 million and 10 million cells. No tumors were detected in the extracellular matrix mice, and no tumors were yet detected in the cells only mice. Before complete conclusions can be drawn about the growth inhibitory potential for extracellular matrix in the presence of cancer cells, tumors will have to appear in the control animals. It is expected that within about 2 weeks tumors will be detected in the control animals. Following the completion of the study the mice will be sacrificed and the tumors will be analyzed histologically.

Criticism of this experiment includes primarily that in this experiment by design the tumor cell inhibition is being studied outside the context of epithelial cancer cells growing in epithelial tissue surrounded by its native extracellular matrix. The tumor cells are injected subcutaneously and therefore grow without a native epithelial matrix to support them. One precept of the invention is that abnormal epithelial cells inhibition will most optimally be demonstrated by contacting the abnormally proliferating cell as it is proliferating at the epithelium of origin. This can not be demonstrated in a subcutaneous model such as tested in this example. Whether inhibition of the subcutaneously implanted human breast cancer cells will occur with in the presence of the extracellular matrix remains to be seen as the data is not complete for these experiments, but the inventor believes that the most optimal demonstration of the efficacy and indeed true nature of the invention will be accomplished by placing a therapeutically effective amount of a composition comprising extracellular matrix at a site where the carcinoma cells are growing in the epithelium of origin. The inhibition of abnormal cell proliferation, and the inhibition of tumor regrowth after resection will be shown when the extracellular matrix is placed at this in vivo site in the actual context of an in situ carcinoma. Local epithelial tissue will be removed or disturbed with the removal or disturbance of abnormally proliferating cells at the site, and contact at this site with extracellular matrix from a mammal will demonstrate growth inhibition, and inhibition of tumor recurrence, as well as general healing of the damaged or diseased tissue.

It is hypothesized that the system may work best in the examples 1-4 that uses tumors generated at the site of origin for the study, so that the tumors can be studied and analyzed in contact with extracellular matrix in the actual context of the epithelial tissues. With these studies the extracellular matrix is facilitating healing and inhibition of abnormal proliferation in the context of epithelial tissue and it is thought by the inventor that this may be a more accurate assessment of the capabilities of the extracellular matrix composition in the animal afflicted with an abnormal cell proliferation disorder.

### Example 6

### Prospective Application of Extracellular Matrix in Humans after Tumor Removal

Surgical candidates are selected from a group of patients diagnosed with carcinoma in various organs or tissues, such as liver, lung, breast, pancreas, ovary, esophagous, skin, colon, prostate, or other organs or tissues in which carcinoma tumors can develop. Those patients deemed eligible for treatment by surgical removal of their tumors and surrounding tissue are prepared for surgery. Removal of the tumor and surrounding tissue is followed by placement of an extracellular matrix material at the site of tumor removal. The form of extracellular matrix material that is used can be sheet matrix, particulate matrix, emulsion, fluidized matrix, or other appropriate form of extracellular matrix. The patient is closed and recovery is monitored non-invasively by MRE and periodic circulating marker analysis. The MRE technology is used to detect recurring carcinoma, scar tissue formation (or lack thereof), and new regenerating tissue at the site of tumor removal.

All references cited are incorporated in their entirety. Although the foregoing invention has been described in detail for purposes of clarity of understanding, it will be obvious that certain modifications may be practiced within the scope of the appended claims.

What is claimed is:

1. A composition comprising a therapeutically effective amount of particulate, emulsion exogenous native extracellular matrix in sufficient amount to contact and fill a wound remaining after surgical tumor excision, or an article comprising a sufficient sheet extracellular matrix to contact and fill a wound remaining after surgical tumor excision.

2. The composition or article of claim 1, wherein said extracellular matrix comprises a layer of extracellular matrix selected from the group consisting of submucosa, basement membrane, and mucosa.

3. The composition or article of claim 1, wherein said extracellular matrix is porcine, bovine or human.

4. The composition or article of claim 1, wherein said extracellular matrix is small intestine submucosa, liver basement membrane, urinary bladder submucosa, or stomach submucosa.

5. A method of inhibiting abnormal proliferation of an epithelial cell in epithelial tissue in a patient afflicted with an epithelial cell proliferation disorder, said method comprising:
a) contacting said epithelial cell with a composition or article comprising soft tissue mammalian extracellular matrix; and
b) detecting resultant inhibition of cell proliferation in said epithelial tissue in said patient.

6. The method of claim 5, wherein said extracellular matrix is liquid or semi-solid, a sheet or a particulate.

7. The method of claim 5, wherein said extracellular matrix is small intestine submucosa, liver basement membrane, urinary bladder submucosa, or stomach submucosa.

8. The method of claim 5, wherein said epithelial cell is malignant.

9. A method of preventing recurrence of an epithelial tumor in a patient comprising;
a) removing at least some of an epithelial tumor from a patient forming a resected tumor site,
b) contacting said resected tumor site with a composition or article comprising mammalian soft tissue extracellular matrix, and
c) monitoring said resected tumor site for tumor recurrence.

10. The method of claim 9, wherein said extracellular matrix is liquid or semi-solid, a sheet or a particulate.

11. The composition or article of claim 9, wherein said extracellular matrix is small intestine submucosa, liver basement membrane, urinary bladder submucosa, or stomach submucosa.

12. The method of claim 9, wherein said extracellular matrix is porcine, bovine or human.
